(19) United States
(12) Patent Application Publication
(10) Pub. No.: US 2016/0089074 A1
(43) Pub. Date: Mar. 31, 2016

(54) VERTEBRA SEGMENTATION APPARATUS, METHOD AND RECORDING MEDIUM

(71) Applicant: FUJIFILM Corporation, Tokyo (JP)

(72) Inventor: Caihua WANG, Tokyo (JP)

(73) Assignee: FUJIFILM Corporation, Tokyo (JP)

(21) Appl. No.: 14/865,867

(22) Filed: Sep. 25, 2015

(30) Foreign Application Priority Data
Sep. 30, 2014 (JP) 2014-200237

(51) Int. Cl. A61B 5/00; G06T 7/00; A61B 6/00; A61B 6/03; A61B 5/055

(52) U.S. Cl. CPC A61B 5/4504; A61B 5/4566; A61B 6/03; A61B 5/055; A61B 6/037; A61B 6/505; A61B 6/5217; G06T 7/004; G06T 7/0079; G06T 2200/04; G06T 2207/10072; G06T 2207/30012

(57) ABSTRACT

An intervertebral foramen position detection unit detects positions of intervertebral foramens in a three-dimensional medical image including plural vertebrae. In this case, for example, a feature value representing a likelihood of an intervertebral foramen is used. A vertebra identification unit identifies each of the plural vertebrae by using the detected positions of the intervertebral foramens, respectively.

### CROSS-REFERENCE TO RELATED APPLICATIONS

The present application claims priority under 35 U.S.C. §119 to Japanese Patent Application No. 2014-200237, filed on Sep. 30, 2014. The application is hereby expressly incorporated by reference, in its entirety, into the present application.

### BACKGROUND

The present disclosure relates to a vertebra segmentation apparatus, method and program that segments plural vertebrae in such a manner that each of the plural vertebrae is recognizable in a three-dimensional medical image including the plural vertebrae.

A spinal cord is an extremely important region that has a function for transmitting messages sent and received between the brain and each region of a body. Therefore, the spinal cord is protected by plural vertebrae (a spinal column). Meanwhile, whether a damage or a lesion is present in a vertebra is checked by reading a tomographic image obtained by scanning a subject. In this case, it is necessary to recognize each vertebra, for example, to report a vertebra in which a damage or a lesion is present. Therefore, various kinds of vertebra segmentation algorithm have been proposed, which are image processing in which plural vertebrae are segmented, based on a tomographic image obtained by scanning a subject, in such a manner that each of the plural vertebrae is recognizable, and a label is attached to each of the vertebrae. For example, Japanese Unexamined Patent Publication No. 2011-131040 (Patent Document 1) proposes a method in which slice images of a plane intersecting and a plane parallel to a center line of each vertebra are generated with respect to a three-dimensional image obtained based on tomographic images, such as a CT (Computed Tomography) image and an MRI (magnetic resonance imaging) image. Further, a feature value representing the clearness of a sectional shape in each of the slice images and a feature value representing regularity of arrangement of vertebrae are calculated, and segmentation into each vertebra is performed by identifying, based on these feature values, the position of an intervertebral disk between vertebrae. Further, labels are attached to vertebra regions after segmentation.

### SUMMARY

Meanwhile, it is generally known that when a slice thickness of a tomographic image in the direction of scan (the direction of the body axis) is large, spatial resolution in the direction of the body axis is insufficient, and image representation performance drops. In the range from the cervical vertebra to an upper part of the thoracic vertebrae, in which the length of each vertebra is relatively short, the thickness of the intervertebral disk is especially small. Therefore, if a slice thickness exceeds, for example, 3 mm, it is difficult to identify the position of the intervertebral disk. As a result, it is impossible to accurately detect the intervertebral disk, and segmentation of plural vertebrae becomes difficult. In this case, a slice thickness may be reduced. However, if the slice thickness is reduced, the data amount of the three-dimensional image becomes extremely large, and an operation amount for segmenting vertebrae also becomes extremely large. Further, if the slice thickness is small, a doctor needs to refer to an even larger number of images during image reading, and a burden on the doctor is heavy.

In view of the foregoing circumstances, the present disclosure provides a vertebra segmentation apparatus, method and program that is able to segment plural vertebrae in such a manner that each of the plural vertebrae is recognizable even if a slice thickness is relatively large.

When vertebrae constituting a spinal column are observed from a side, in two adjacent vertebrae, an intervertebral foramen is formed by a notch on the upper vertebra (an inferior vertebral notch) and a notch on the lower vertebra (a superior vertebral notch) facing each other. The intervertebral foramen is a path of spinal nerves from a spinal cord in a spinal canal. When this intervertebral foramen is observed from a side (in other words, in a sagittal slice plane), plural vertebrae are periodically present in the direction in which the spinal column extends, and the size of an intervertebral foramen in the direction in which the spinal column extends is slightly larger than the thickness of an intervertebral disk. The inventor of the present disclosure has reached the present disclosure by noting this feature.

Specifically, a vertebra segmentation apparatus of the present disclosure includes an intervertebral foramen position detection means that detects positions of intervertebral foramens in a three-dimensional medical image including plural vertebrae, and a vertebra identification means that identifies each of the plural vertebrae by using the detected positions of the intervertebral foramens, respectively.

The expression “detects positions of intervertebral foramens” means detecting the position of a voxel belonging to the intervertebral foramen in the three-dimensional medical image.

In the vertebra segmentation apparatus of the present disclosure, the intervertebral foramen position detection means may detect the positions of the intervertebral foramens by using feature values representing a likelihood of an intervertebral foramen in the three-dimensional medical image.

As the feature value representing a likelihood of an intervertebral foramen, an output from a classifier created to detect an intervertebral foramen may be used. Alternatively, a voxel value representing an intervertebral foramen in a three-dimensional medical image may be used.

The vertebra segmentation apparatus of the present disclosure may further include a center line detection means that detects at least one of a center line of a spinal cord and a center line of a spinal column in the three-dimensional medical image, a slice image generation means that generates, with respect to at least one of the center line of the spinal cord and the center line of the spinal column, at least one slice image for detecting the intervertebral foramens, and a feature value calculation means that calculates the feature value representing a likelihood of an intervertebral foramen based on the at least one slice image. Further, the intervertebral foramen position detection means may detect the positions of the intervertebral foramens based on the feature value.

In the vertebra segmentation apparatus of the present disclosure, the slice image generation means may generate plural slice images about plural slice planes at a predetermined interval that are orthogonal to the center line of the spinal cord or the center line of the spinal column.

The “predetermined interval” is determined while an operation speed and detection accuracy are taken into consideration. For example, a value of about 3 to 7 mm, and desirably a value of about 5 mm may be used.

In the vertebra segmentation apparatus of the present disclosure, the feature value calculation means may calculate a first feature value representing a likelihood of an intervertebral foramen in one side of each of the plural slice images and a second feature value representing a likelihood of an intervertebral foramen in the other side of each of the plural slice images, when each slice image is divided in such a manner that the one side and the other side are symmetric with respect to a straight line passing through the center line of the spinal cord or the center line of the spinal column, and determine a representative value of the first feature value and the second feature value, as the feature value representing a likelihood of an intervertebral foramen.

Further, in the vertebra segmentation apparatus of the present disclosure, the slice image generation means may generate the at least one slice image of a slice plane that includes the center line of the spinal cord or the center line of the spinal column and faces a predetermined direction.

The “predetermined direction” is a direction with respect to a center line of a spinal cord or a center line of a spinal column, and a probability of presence of an intervertebral foramen in the direction is high. For example, when the center line of the spinal cord is used, if a line that passes through the center line of the spinal cord and extends in the transverse direction of the body from the left to the right is used as a base line, a direction at 30 to 45 degrees, with respect to the base line, viewed from the center line of the spinal cord may be used as the predetermined direction.

Further, in the vertebra segmentation apparatus of the present disclosure, the intervertebral foramen position detection means may fit the feature value to a predetermined periodic function or quasiperiodic function in the direction of the center line of the spinal cord or the center line of the spinal column, and detect the position of each of the intervertebral foramens based on the periodic function or the quasiperiodic function to which the feature value has been fitted.

Further, in the vertebra segmentation apparatus of the present disclosure, the feature value calculation means may calculate a first feature value representing a likelihood of an intervertebral foramen in one side of each of the plural slice images and a second feature value representing a likelihood of an intervertebral foramen in the other side of each of the plural slice images, when each slice image is divided in such a manner that the one side and the other side are symmetric with respect to a straight line passing through the center line of the spinal cord or the center line of the spinal column. Further, the intervertebral foramen position detection means may fit each of the first feature value and the second feature value to a predetermined periodic function or quasiperiodic function in the direction of the center line of the spinal cord or the center line of the spinal column, and detect the position of each of the intervertebral foramens based on the periodic function or the quasiperiodic function to which each of the first feature value and the second feature value has been fitted.

In the vertebra segmentation apparatus of the present disclosure, the intervertebral foramen position detection means may detect the position of each of the intervertebral foramens also by using a feature value representing clearness of a slice plane intersecting the center line of the spinal cord or the center line of the spinal column, a feature value representing clearness of a slice plane parallel to the center line of the spinal cord or the center line of the spinal column and feature values representing regularity of the arrangement of the vertebrae that are calculated based on sharpness of the intersecting slice plane and sharpness of the parallel slice plane.

A vertebra segmentation method of the present disclosure includes the steps of detecting positions of intervertebral foramens in a three-dimensional medical image including plural vertebrae, and identifying each of the plural vertebrae by using the detected positions of the intervertebral foramens, respectively.

A vertebra segmentation program of the present disclosure may be provided as a program that causes a computer to execute the vertebra segmentation method.

According to the present disclosure, the positions of intervertebral foramens are detected in a three-dimensional medical image including plural vertebrae, and each of the plural vertebrae is identified by using the detected positions of the intervertebral foramens, respectively. Here, when a spinal column is observed from a side, the size of an intervertebral foramen in the longitudinal direction of the spinal column is slightly larger than the thickness of an intervertebral disk. Therefore, even if a slice thickness of a three-dimensional medical image is relatively large, the position of an intervertebral foramen is detectable. Consequently, it is possible to perform segmentation of plural vertebrae in such a manner that each of the plural vertebrae is recognizable, and to identify each of the plural vertebrae.

### BRIEF DESCRIPTION OF THE DRAWINGS

FIG. 1 is a schematic diagram illustrating the hardware configuration of a diagnosis-assistance system to which a vertebra segmentation apparatus according to a first embodiment of the present disclosure has been applied;

FIG. 2 is a schematic diagram illustrating the configuration of a vertebra segmentation apparatus achieved by installing a vertebra segmentation program in a computer;

FIG. 3 is a diagram for explaining detection of a center line of a spinal cord;

FIG. 4 is a schematic diagram illustrating plural slice images that will be generated;

FIG. 5 is a diagram illustrating an example of a slice image;

FIG. 6 is a diagram illustrating sample images including an intervertebral foramen;

FIG. 7 is a diagram illustrating an example of calculating a feature value;

FIG. 8 is a diagram for explaining detection of the positions of intervertebral foramens;

FIG. 9 is a diagram for explaining a change in feature value along a center line of a spinal cord;

FIG. 10 is a diagram for explaining processing for identifying vertebrae;

FIG. 11 is a schematic diagram illustrating a sagittal image representing arrangement of vertebrae;

FIG. 12 is a flow chart showing processing performed in the first embodiment;

FIG. 13 is a diagram for explaining detection of the position of an intervertebral foramen in a third embodiment; and

FIG. 14 is a diagram for explaining detection of the position of an intervertebral foramen in a third embodiment.

### DESCRIPTION OF THE PREFERRED EMBODIMENTS

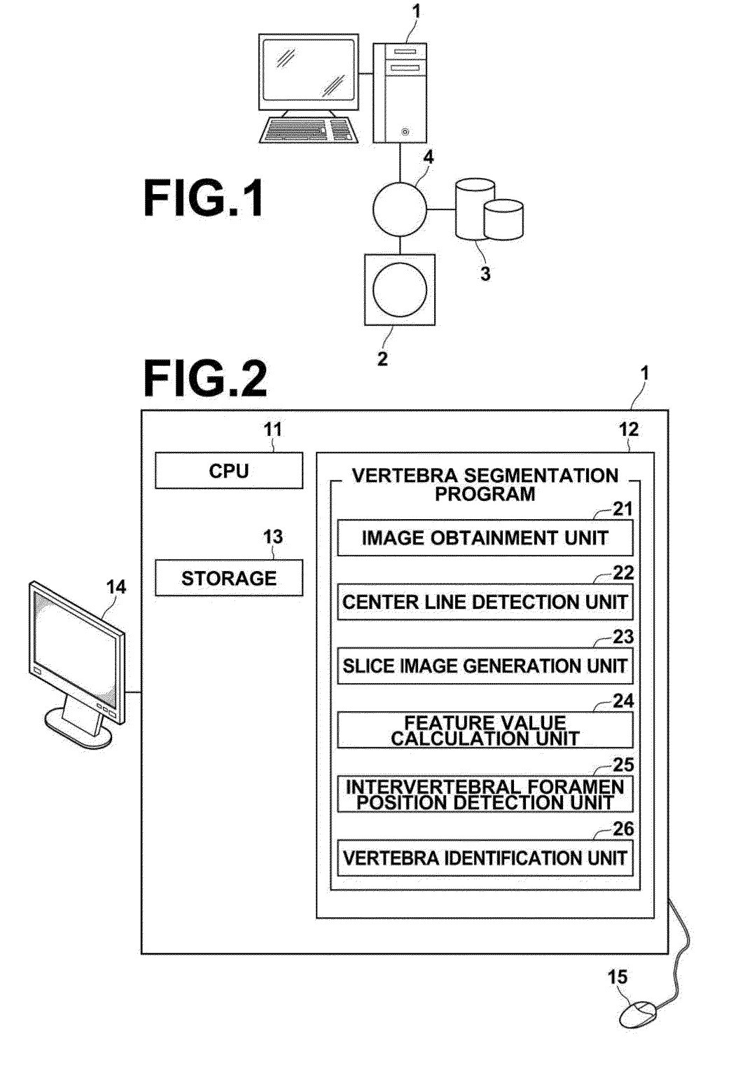

Hereinafter, embodiments of the present disclosure will be described with reference to drawings. FIG. 1 is a schematic diagram illustrating the hardware configuration of a diagnosis-assistance system to which a vertebra segmentation apparatus according to a first embodiment of the present disclosure has been applied. As illustrated in FIG. 1, this system includes a vertebra segmentation apparatus 1 according to the first embodiment, a three-dimensional image imaging apparatus 2 and an image storage server 3, which are connected to each other through a network 4 in such a manner that they can communicate with each other.

The three-dimensional image imaging apparatus 2 generates a three-dimensional image representing a region of a subject that is a target of diagnosis by imaging the region. Specifically, the three-dimensional image imaging apparatus 2 is a CT apparatus, an MRI apparatus, a PET (Positron Emission Tomography) apparatus and the like. A three-dimensional image generated by this three-dimensional image imaging apparatus 12 is sent to the image storage server 3, and stored in the image storage server 3. In the present embodiment, it is assumed that the diagnosis target region of the subject is vertebrae, and the three-dimensional image imaging apparatus 2 is a CT apparatus, and the three-dimensional is a CT image.

The image storage server 3 is a computer that stores and manages various kinds of data, and includes a large capacity external storage unit and software for managing a database. The image storage server 3 sends and receives image data or the like by communicating with other apparatuses through the wired or wireless network 4. Specifically, the image storage server 3 receives image data, such as a three-dimensional image generated at the three-dimensional image imaging apparatus 2, through the network, and stores the image data in a recording medium, such as the large capacity external storage unit, and manages the image data. Here, the storage format of image data and communication between apparatuses through the network 4 are based on a protocol of DICOM (Digital Imaging and Communication in Medicine) or the like. Further a tag based on DICOM standard is attached to the three-dimensional image. The tag includes a patient's name, information representing an imaging apparatus, a date and time of imaging and information about an imaged region.

The vertebra segmentation apparatus 1 is a computer in which a vertebra segmentation program of the present disclosure has been installed. The computer may be a workstation or a personal computer directly operated by a doctor who makes a diagnosis, or a server computer connected to them through a network. The vertebra segmentation program is recorded in a recording medium, such as a DVD and a CD-ROM, and distributed. The vertebra segmentation program is installed in a computer from the recording medium. Alternatively, the vertebra segmentation program is stored in a storage unit of a server computer connected to a network or in a network storage in an accessible state from the outside, and downloaded based on a request and installed in a computer used by a doctor.

FIG. 2 is a schematic diagram illustrating the configuration of a vertebra segmentation apparatus achieved by installing a vertebra segmentation program in a computer. As illustrated in FIG. 2, the vertebra segmentation apparatus 1 includes a CPU 11, a memory 12 and a storage 13, as the standard configuration of a workstation. Further, a display 14 and an input unit 15, such as a mouse, are connected to the vertebra segmentation apparatus 1.

A three-dimensional image obtained from the image storage server 3 through the network 4, an image generated in processing at the vertebra segmentation apparatus 1, and various kinds of information including information necessary for processing have been stored in the storage 13.

Further, the memory 12 stores a vertebra segmentation program. The vertebra segmentation program defines, as processing performed by the CPU 11, image obtainment processing, center line detection processing, slice image generation processing, feature value calculation processing, intervertebral foramen position detection processing, and vertebra identification processing. In the image obtainment processing, three-dimensional image V1 of a subject including plural vertebrae, as a diagnosis target, and which was obtained by the three-dimensional image imaging apparatus 2, is obtained. In the center line detection processing, at least one of a center line of a spinal cord and a center line of a spinal column is detected in three-dimensional image V1. In the slice image generation processing, at least one slice image for detecting an intervertebral foramen is generated with respect to at least one of the center line of the spinal cord and the center line of the spinal column. In the feature value calculation processing, a feature value representing a likelihood of an intervertebral foramen is calculated based on the at least one slice image. In the intervertebral foramen position detection processing, the position of an intervertebral foramen is detected by using the feature value representing a likelihood of an intervertebral foramen. In the vertebra identification processing, each of plural vertebrae is identified by using the detected intervertebral foramens, respectively.

Then, when these kinds of processing are executed by the CPU 11 according to the program, a computer functions as an image obtainment unit 21, a center line detection unit 22, a slice image generation unit 23, a feature value calculation unit 24, an intervertebral foramen position detection unit 25, and a vertebra identification unit 26. Here, the vertebra segmentation apparatus 1 may include plural CPU's that perform image obtainment processing, center line detection processing, slice image generation processing, feature value calculation processing, intervertebral foramen position detection processing, and vertebra identification processing, respectively.

The image obtainment unit 21 obtains three-dimensional image V1 from the image storage server 3. The image obtainment unit 21 may obtain three-dimensional image V1 from the storage 13 when three-dimensional image V1 has been already stored in the storage 13. In the present embodiment, it is assumed that the direction of a body axis of three-dimensional image V1 is set as z-axis, a direction from the dorsal side to the ventral side of the subject in three-dimensional image V1 is set as x-axis, and a transverse direction from the left to the right of the subject is set as y-axis.

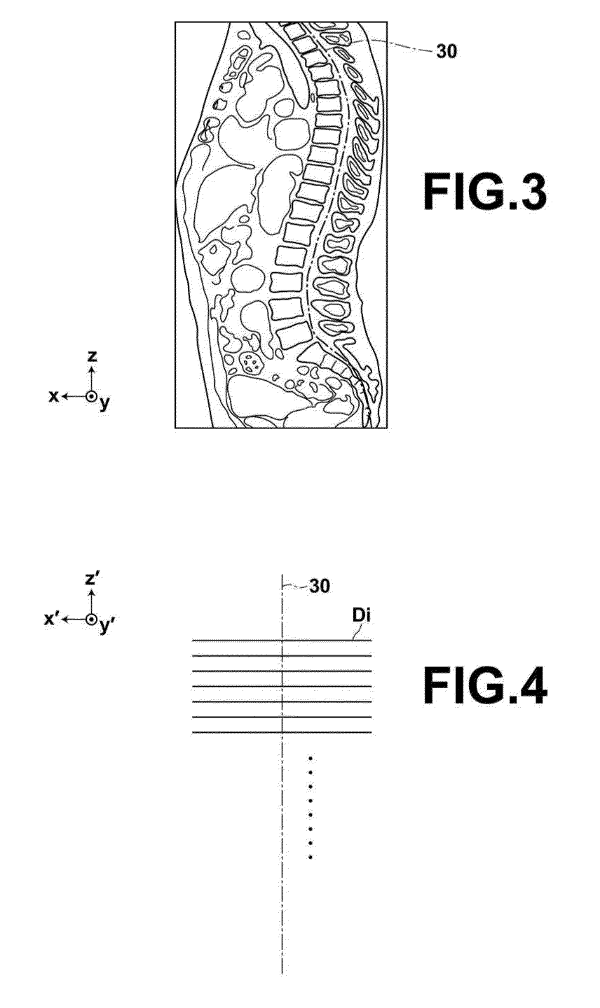

The center line detection unit 22 detects at least one of the center line of the spinal cord and the center line of the spinal column in three-dimensional image V1. In the present embodiment, it is assumed that the center line of the spinal cord is detected. As a method for detecting the center line of the spinal cord, for example, a method disclosed in Japanese Unexamined Patent Publication No. 2011-142960 is used. In the method disclosed in Japanese Unexamined Patent Publication No. 2011-142960, plural slice images of axial slice planes, which are orthogonal to the body axis, are generated based on three-dimensional image V1. Further, sectional shapes of the spinal cord are detected in the plural slice images, and the center line 30 of the spinal cord, as illustrated in FIG. 3, is detected by interpolating positions between the positions of the detected plural sectional shapes. The method for detecting the center line of the spinal cord or the center line of the spinal column is not limited to this method. An arbitrary method, for example, such as a method disclosed in Japanese Unexamined Patent Publication No. 2009-207886, may be used.

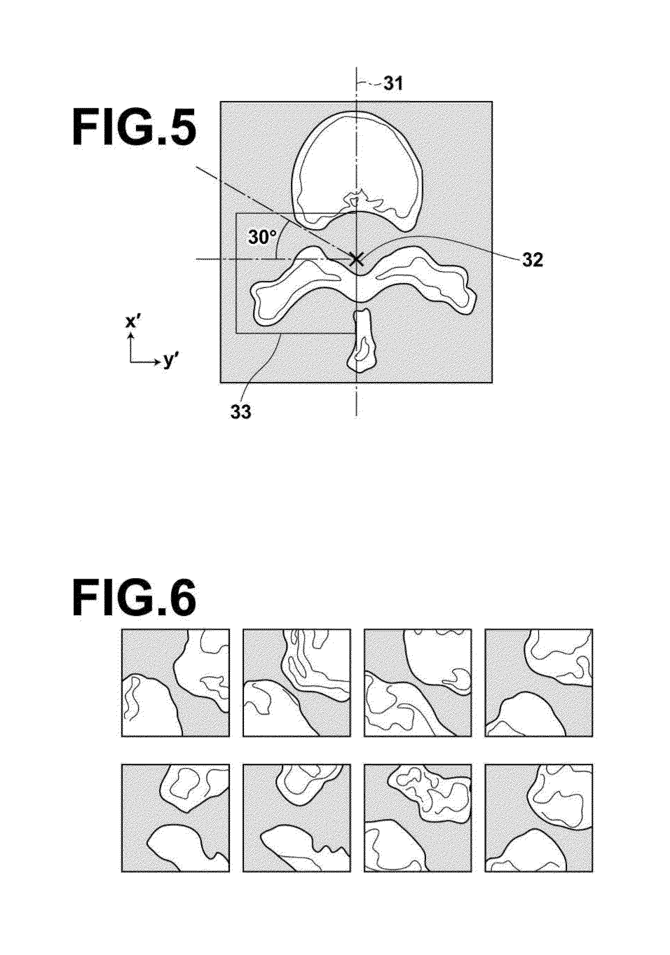

The slice image generation unit 23 generates, with respect to at least one of the center line of the spinal cord and the center line of the spinal column, at least one slice image for detecting an intervertebral foramen. In the present embodiment, the slice image generation unit 23 generates, with respect to the center line 30 of the spinal cord, plural slice images Di (i=1 through n, n is the number of slice images) orthogonal to the center line 30 of the spinal cord. The slice image generation unit 23 generates, based on three-dimensional image V1, plural slice images Di along the center line 30 of the spinal cord at a predetermined interval (for example, an interval of 3 to 7 mm, and in the present embodiment, an interval of 5 mm). Specifically, the slice image generation unit 23 transforms xyz coordinate system of three-dimensional image V1 into x′y′z′ coordinate system in which the center line 30 of the spinal cord is set as z′ axis. Further, the slice image generation unit 23 generates plural slice images Di orthogonal to z′ axis in the x′y′z′ coordinate system after transformation. FIG. 4 is a schematic diagram illustrating plural slice images that will be generated. As illustrated in FIG. 4, slice images Di generated by the slice image generation unit 23 are present on plural planes that are orthogonal to z′ axis, i.e., the center line 30 of the spinal cord and parallel to each other. In other words, slice images Di are present on slice planes orthogonal to the center line 30 of the spinal cord. FIG. 5 is a diagram illustrating an example of a slice image. It depends on the position of the slice plane of a slice image, but the slice image represents a vertebra that is cut at a slice plane orthogonal to the center line 30 of the spinal cord, as illustrated in FIG. 5.

It is possible to generate a three-dimensional image by arranging plural slice images Di along the center line 30 of the spinal cord. Hereinafter, three-dimensional image V1 obtained by the three-dimensional image imaging apparatus 2 will be referred to as first three-dimensional image V1. A three-dimensional image generated by arranging plural slice images Di along the center line 30 of the spinal cord will be referred to as second three-dimensional image V2. The coordinate system of second three-dimensional image V2 is x′y′z′ coordinate system.

The feature value calculation unit 24 calculates a feature value representing a likelihood of an intervertebral foramen. First, the feature value calculation unit 24 sets, in each slice image Di, a search range 33 with respect to an intersection 32 with the center line 30 of the spinal cord. In the present embodiment, a line that passes through the intersection 32 and extends in the direction of x′ axis is set as a center line 31, as illustrated in FIG. 5. The center line 31 divides slice image Di into two regions in such a manner that the two regions are symmetric with respect to the center line 31. The feature value calculation unit 24 sets a rectangular search range 33 in a region on the left side of the center line 31 in such a manner that the rectangular search range 33 has one of its sides on the center line 31 and includes a transverse process of the spinal column. Here, the search range may be set in a region on the right side of the center line 31.

Here, the feature value calculation unit 24 includes a classifier for calculating a feature value representing a likelihood of an intervertebral foramen. The classifier is obtained by performing machine learning on plural sample images including intervertebral foramens on slice planes orthogonal to the center line 30 of the spinal cord, as illustrated in FIG. 6, for example, by using a method, such as AdaBoost algorithm. Then, the feature value calculation unit 24 applies the classifier to a two-dimensional patch having the same size as the sample image in slice image Di, and calculates, as the feature value representing a likelihood of an intervertebral foramen, the maximum value of outputs from the classifier in the search range. The feature value calculation unit 24 obtains feature values in the search range, as illustrated in FIG. 5, by calculating feature values in a predetermined range of angles clockwise from a line that passes through the intersection 32 and is orthogonal to the center line 31. As the predetermined range of angles, a range of 20 degrees to 45 degrees, and desirably a range of 30 degrees may be used.

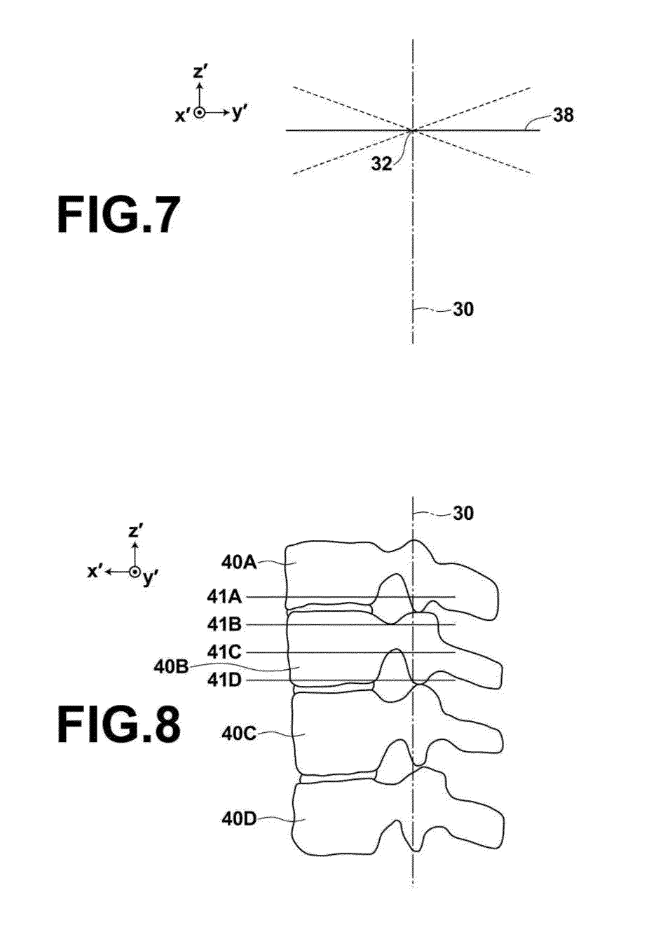

Alternatively, the feature value calculation unit 24 may set, in second three-dimensional image V2, a plane 38 having its center at the intersection 32 with the center line 30 of the spinal cord in each slice image Di, as illustrated in FIG. 7. The feature value calculation unit 24 may three-dimensionally incline this plane 38 within a predetermined range of angles with respect to x′y′ plane. Further, the feature value calculation unit 24 may calculate a feature value by using voxel values of second three-dimensional image V2 on the inclined plane. Consequently, even if a center position of the intervertebral foramen is not present in slice image Di, it is possible to widen the search range of the intervertebral foramen. Therefore, it is possible to more definitely calculate the feature value representing a likelihood of an intervertebral foramen. It is desirable that the range of angles is, for example, about ±20 degrees.

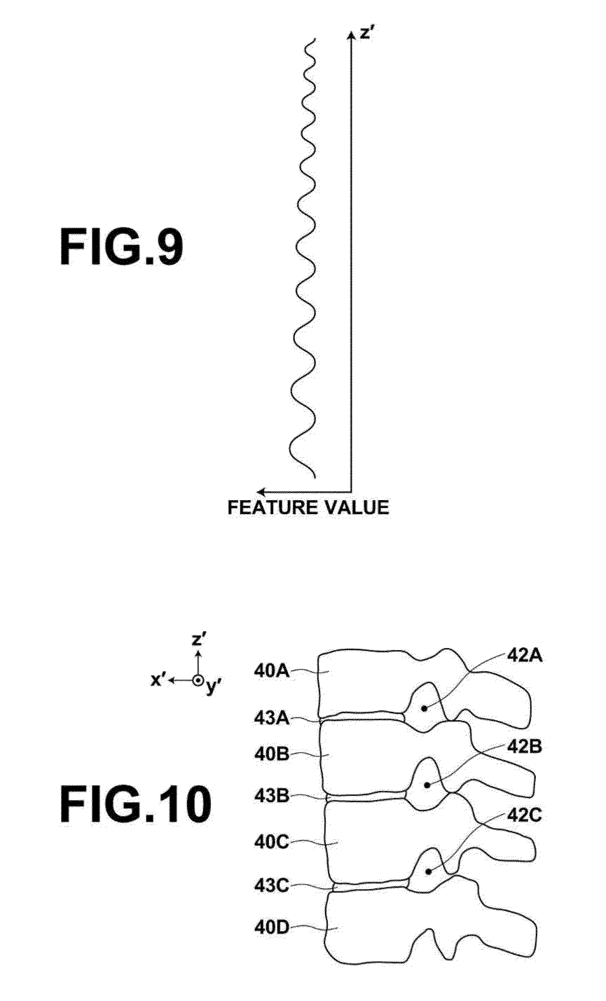

The intervertebral foramen position detection unit 25 detects the position of the intervertebral foramen based on the feature value calculated by the feature value calculation unit 24. FIG. 8 is a diagram for explaining detection of the positions of the intervertebral foramens. In FIG. 8, a part of the spinal column in x′y′z′ coordinate system is schematically illustrated. FIG. 8 illustrates four vertebrae 40A through 40D and four slice planes 41A through 41D orthogonal to the center line 30 of the spinal cord. Slice plane 41A passes through an approximate center of an intervertebral foramen. Therefore, a feature value calculated in the slice image of slice plane 41A is a relatively large value. Slice plane 41B does not pass through any intervertebral foramen. Therefore, a feature value calculated in the slice image of slice plane 41B is a relatively small value. Slice plane 41C only slightly passes through an intervertebral foramen. Therefore, a feature value calculated in the slice image of slice plane 41C is a relatively small value. Slice plane 41D passes through an approximate center of an intervertebral foramen. Therefore, a feature value calculated in the slice image of slice plane 41D is a relatively large value. Therefore, when the values of the feature values are plotted along the direction of the center line 30 of the spinal cord, and smoothly connected to each other, a curve periodically changes along z′ axis, as illustrated in FIG. 9, is obtained. The feature values have a local maximum at the position of an intervertebral foramen, and a local minimum at a position between intervertebral foramens. The intervertebral foramen position detection unit 25 plots the values of feature values along the direction of the center line 30 of the spinal cord, and generates a curve that periodically changes by smoothly connecting plots. Further, the intervertebral foramen position detection unit 25 detects, as the position of an intervertebral foramen, the position of a voxel in second three-dimensional image V2 at which the feature value has a local maximum in the curve. Here, the detected position of the intervertebral foramen represents the center of the intervertebral foramen.

The vertebra identification unit 26 identifies each of plural vertebrae by using the positions of the intervertebral foramens detected by the intervertebral foramen position detection unit 25. FIG. 10 is a diagram for explaining processing for identifying vertebrae. In FIG. 10, a part of the spinal column in x′y′z′ coordinate system is schematically illustrated. In FIG. 10, four vertebrae 40A through 40D and centers 42A through 42C of three intervertebral foramens have been detected. As illustrated in FIG. 10, intervertebral disks 43A through 43C are present between vertebrae 40A through 40D. Meanwhile, anatomical positional relationships between centers 42A through 42C of intervertebral foramens and intervertebral disks 43A through 43C, respectively, are fixed. Therefore, The vertebra identification unit 26 identifies, based on an anatomical positional relationship between the position of an intervertebral foramen and the position of an intervertebral disk, the position of the intervertebral disk by using the position of the intervertebral foramen detected in second three-dimensional image V2. The position of the intervertebral disk in z′ axis is sufficient as the position of the intervertebral disk. Therefore, the identified position of the intervertebral disk includes only a value on z′ axis.

Then, the vertebra identification unit 26 transforms the identified position of the intervertebral disk into the coordinate system of first three-dimensional image V1. Such coordinate transformation is easy, because a positional relationship between a point on z′ axis and xyz coordinate of first three-dimensional image V1 is known. Accordingly, the position of the intervertebral disk in z direction in the coordinate system of first three-dimensional image V1 is identified. Meanwhile, vertebrae and intervertebral disks are alternately present in the spinal column. Therefore, the vertebra identification unit 26 identifies each of vertebrae by performing, based on the identified intervertebral disks, segmentation of plural vertebrae in such a manner that the vertebrae are recognizable.

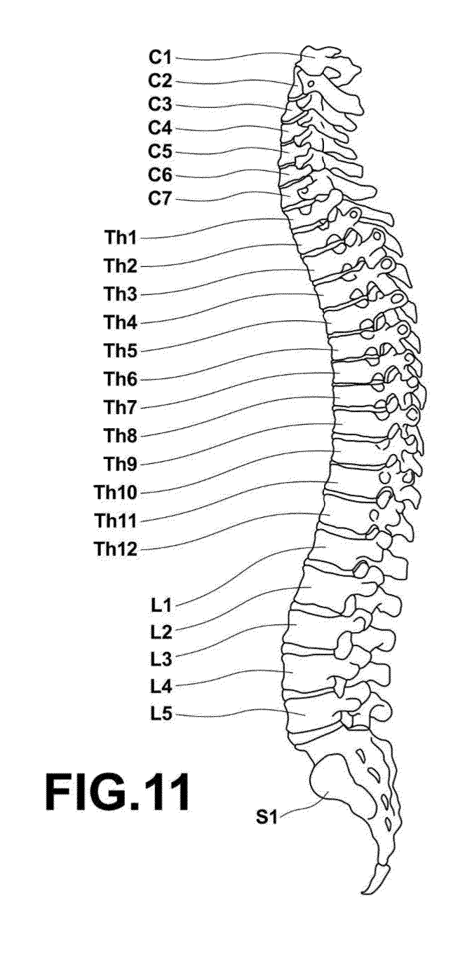

The vertebra identification unit 26 attaches a label to each of the identified vertebrae. In the present embodiment, anatomical types of vertebrae are used as labels. FIG. 11 is a schematic diagram illustrating a sagittal image showing arrangement of vertebrae. As illustrated in FIG. 11, anatomical numbers are assigned to vertebrae. The spinal column includes four parts of cervical vertebrae, thoracic vertebrae, lumbar vertebrae and a sacrum. The cervical vertebrae include first through seventh cervical vertebrae, and anatomically, identification information C1 through C7 is attached to the first through seventh cervical vertebrae, respectively. The thoracic vertebrae include first through 12th thoracic vertebrae, and anatomically, identification information Th1 through Th12 is attached to the first through 12th thoracic vertebrae, respectively. The lumbar vertebrae include first through fifth lumbar vertebrae, and anatomically, identification information L1 through L5 is attached to the first through fifth lumbar vertebrae, respectively. The sacrum includes only one bone, and anatomically, identification information S1 is attached to the sacrum. The vertebra identification unit 26 attaches these pieces of identification information to each of identified vertebrae, respectively, as labels.

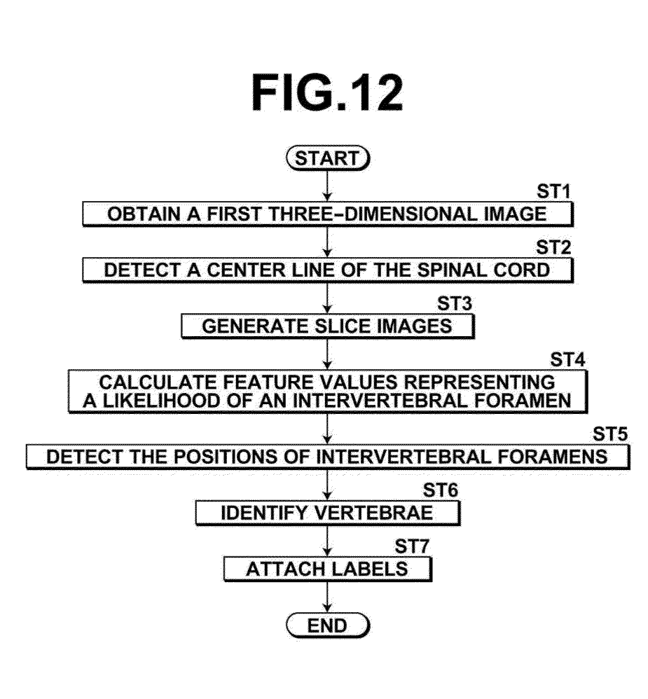

Next, processing performed in the present embodiment will be described. FIG. 12 is a flow chart showing processing performed in the present embodiment. First, the image obtainment unit 21 obtains first three-dimensional image V1, which is a diagnosis target, from the image storage server 3 (step ST1). The center line detection unit 22 detects the center line 30 of the spinal cord (step ST2). Then, the slice image generation unit 23 generates plural slice images Di orthogonal to the center line 30 of the spinal cord (step ST3). The feature value calculation unit 24 calculates feature values representing a likelihood of an intervertebral foramen based on plural slice images Di (step ST4).

Then, the intervertebral foramen position detection unit 25 detects the positions of intervertebral foramens based on the feature values representing a likelihood of an intervertebral foramen (step ST5). Further, the vertebra identification unit 26 identifies vertebrae based on the detected positions of intervertebral foramens (step ST6), and attaches a label to each of the identified vertebrae (step ST7), and ends processing.

As described above, in the first embodiment, the positions of intervertebral foramens are detected in a three-dimensional medical image including plural vertebrae, and the plural vertebrae are identified by using the detected positions of the intervertebral foramens. When the spinal column is observed from a side, the size of an intervertebral foramen in the longitudinal direction of the spinal column is slightly larger than the thickness of an intervertebral disk. Therefore, even if a slice thickness of the three-dimensional image is relatively large, it is possible to detect the positions of intervertebral foramens. As a result, it is possible to segment the plural vertebrae in a recognizable manner, and to identify each of the vertebrae. Therefore, it is possible to attach labels to the vertebrae, as illustrated in FIG. 11.

Next, a second embodiment of the present disclosure will be described. The second embodiment differs from the first embodiment only in processing for detecting the position of an intervertebral foramen. Therefore, detailed explanations about the configuration of the apparatus will be omitted here.

As described above, intervertebral foramens are periodically present in the direction of the center line 30 of the spinal cord. In the second embodiment, the positions of intervertebral foramens are detected by fitting feature values representing a likelihood of an intervertebral foramen calculated by the feature value calculation unit 24 to a predetermined fitting function (a periodic function or a quasiperiodic function). Here, the “periodic function” is a function having periodicity about positions on z′ axis, and the cycle of which is constant regardless of positions on z′ axis. Further, the “quasiperiodic function” is a function having periodicity about positions on z′ axis, but the cycle of which fluctuates, depending on positions on z′ axis.

As the fitting function, a periodic function, such as trigonometric functions, may be used. However, plural vertebrae have structural characteristics that the height of a vertebra in the direction of z′ axis gradually becomes larger from the cervical vertebrae through the lumbar vertebrae. Therefore, in the present embodiment, quasiperiodic function g(z′) represented by the following expression (1) is used as a fitness function. This fitness function has been stored in the storage 13.

$$\begin{matrix}[ Expression 1 ] & \\ g ( {z}^{′} ) = cos \frac{2 π ( {z}^{′} - c )}{{az}^{′} + b} . & ( 1 )\end{matrix}$$

In expression (1), a, b and c are coefficients for determining the shape of g(z′). Function g(z′) is a periodic function when a=0.

The definition of values of this expression g(z′) agrees with the definition of values of a feature value representing a likelihood of an intervertebral foramen. Specifically, expression g(z′) has a local maximum at the center of an intervertebral foramen and a local minimum at a position between adjacent intervertebral foramens.

Further, the intervertebral foramen position detection unit 25 globally fits feature values representing a likelihood of an intervertebral foramen. Here, the expression “globally fits” means fitting feature values in the whole possible range at which position z′ may be present on z′ axis. The intervertebral foramen position detection unit 25 may determine optimal coefficients a, b and c by performing multivariable analysis, such as a method of least squares. For example, evaluation value H of fitting is represented by the following expression (2). Here, f(z′) is a feature value representing a likelihood of an intervertebral foramen.

$$\begin{matrix}[ Expression 2 ] & \\ H ( a , b , c ) = {\sum }_{{z}^{′} = 1}^{{z}^{′}} f ( {z}^{′} ) cos \frac{2 π ( {z}^{′} - c )}{{az}^{′} + b} . & ( 2 )\end{matrix}$$

Coefficients a, b and c are selected so that this evaluation value H is maximized. In this case, ranges of possible values of coefficients a, b and c are determined in advance, and all the combinations of a, b and c in the ranges are searched. Accordingly, it is possible to fit the feature values representing a likelihood of an intervertebral foramen, and which have been calculated by the feature value calculation unit 24, to the fitting function represented by expression (1).

The intervertebral foramen position detection unit 25 detects the position of each of intervertebral foramens based on the fitting function to which the feature value has been fitted. For example, in the example of the fitting function represented by expression (1), position z′(n) of the n-th intervertebral foramen may be obtained as in the following expression (3):

$$\begin{matrix}[ Expression 3 ] & \\ {z}^{′} ( n ) = \frac{2 c + ( 2 n + 1 ) b}{2 c - ( 2 n + 1 ) a} . & ( 3 )\end{matrix}$$

Here, if an intervertebral foramen has been flattened by an injury or a disease, it is impossible to detect the position of the intervertebral foramen by using the feature value of an intervertebral foramen alone. In the second embodiment, the feature value representing a likelihood of an intervertebral foramen is fitted to a fitting function. Therefore, it is possible to detect periodic presence of intervertebral foramens. Hence, it is possible to more accurately detect the position of the intervertebral foramen.

Next, a third embodiment of the present disclosure will be described. The third embodiment differs from the first embodiment only in processing for detecting the position of an intervertebral foramen. Therefore, detailed explanations about the configuration of the apparatus will be omitted here.

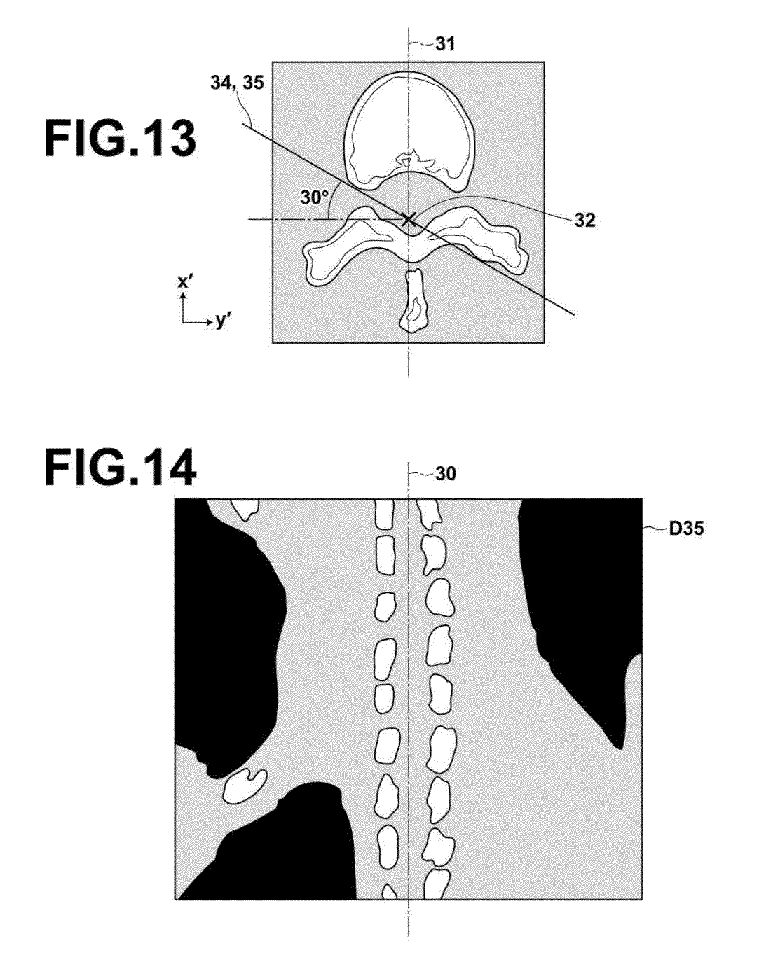

FIG. 13 is a diagram for explaining detection of the position of an intervertebral foramen in the third embodiment. In the third embodiment, the intervertebral foramen position detection unit 25 sets, in one of slice images Di, a straight line 34 that passes through an intersection 32 with the center line 30 of the spinal cord and inclines at a predetermined angle with respect to y′ axis. Further, the intervertebral foramen position detection unit 25 sets, in second three-dimensional image V2, a slice plane 35 that passes through the straight line 34 and is parallel to z′ axis, and generates slice image D35 on the slice plane 35. Here, it is desirable that the predetermined angle is 30 degrees through 45 degrees. In the present embodiment, the predetermined angle is 30 degrees. Here, the straight line 34 passes through a position in an intervertebral foramen in slice image Di. Therefore, in second three-dimensional image V2, the slice plane 35 cuts plural intervertebral foramens. Meanwhile, the region of a vertebra has a high CT value in slice image D35. Therefore, in slice image D35, the region of intervertebral foramens having low CT values clearly appears between regions of high CT values, as illustrated in FIG. 14.

Therefore, in the third embodiment, the intervertebral foramen position detection unit 25 detects the position of an intervertebral foramen in slice image D35. Specifically, the intervertebral foramen position detection unit 25 binarizes a region on the left side of the center line 30 of the spinal cord in slice image D35, and removes a noise composed of a low CT value included in a region having a high CT value by performing a morphology operation on the region having the high CT value. Accordingly, the intervertebral foramen position detection unit 25 extracts the region having the high CT value. In second three-dimensional image V2, the region having the high CT value is a bone region, and the region having the low CT value between the regions having the high CT value is an intervertebral foramen region. Therefore, the intervertebral foramen position detection unit 25 uses, as a feature value representing a likelihood of an intervertebral foramen, a CT value lower than a predetermined threshold, and detects, as the position of an intervertebral foramen, the position of a region having a low CT value between the extracted regions having high CT values.

In the third embodiment, with respect to slice image D35, a classifier for calculating a feature value representing a likelihood of an intervertebral foramen based on slice image D35 may be prepared in a similar manner to the first embodiment. Further, an output from the classifier may be calculated as the feature value representing a likelihood of an intervertebral foramen, and the position of the intervertebral foramen may be detected based on the calculated feature value representing a likelihood of an intervertebral foramen. In this case, the position of an intervertebral foramen may be detected by fitting the calculated feature value to a fitting function in a similar manner to the second embodiment.

Next, a fourth embodiment of the present disclosure will be described. The fourth embodiment differs from the first embodiment only in processing for detecting the position of an intervertebral foramen. Therefore, detailed explanations about the configuration of the apparatus will be omitted here.

In the first embodiment, the feature value calculation unit 24 calculates a feature value representing a likelihood of an intervertebral foramen, and detects the position of an intervertebral foramen based on the feature value representing a likelihood of an intervertebral foramen. In the fourth embodiment, the position of the intervertebral foramen is detected further by using another feature value.

In the fourth embodiment, the feature value calculation unit 24 calculates a feature value representing the clearness of a sectional shape orthogonal to the center line 30 of the spinal cord, in other words, z′ axis (which will be referred to as orthogonal slice plane feature value fl(z′)) and a feature value representing the clearness of plural sectional shapes parallel to the center line 30 of the spinal cord (which will be referred to as parallel slice plane feature value 12(z′)) in a manner similar to a method, for example, disclosed in Patent Document 1. As orthogonal slice plane feature value f1(z), for example, a feature value for extracting a ring-shaped pattern having a predetermined point on z′ axis, as a center, is used. This feature value is calculated, for example, by using an eigenvalue analysis method of the Hessian matrix. Here, the predetermined point may be a point on slice image Di in the first embodiment. As parallel slice plane feature value f2(z′), for example, a feature value for extracting a tubular pattern that extends in the direction of z′ axis and has a predetermined point on z′ axis, as a center, is used. This feature value is calculated, for example, by using an eigenvalue analysis method of the Hessian matrix. Further, the feature value calculation unit 24 calculates a classifier output feature value, which is an output from a classifier for calculating a feature value representing a likelihood of an intervertebral foramen, (which will be referred to as f3(z′)) in a similar manner to the first embodiment. Classifier output feature value f3(z′) in the fourth embodiment is the same value as the feature value representing a likelihood of an intervertebral foramen in the first and second embodiments.

Then, the feature value calculation unit 24 calculates feature value f(z) representing a likelihood of an intervertebral foramen by the following expression (4) by using orthogonal slice plane feature value f1(z′), parallel slice plane feature value f2(z′) and classifier output feature value f3(z′). In expression (4), a is a weighting coefficient and G(z′,σ) is a Gaussian function using standard deviation as σ.

$$\begin{matrix}[ Expression 4 ] & \\ f ( {z}^{′} ) = f 1 ( {z}^{′} ) + α {max}_{σ ∈ [ σ 0 , σ 1 ]} [ \frac{{d}^{2} G ( {z}^{′} , σ )}{d {z}^{′2}} ⊗ f 2 ( {z}^{′} ) ] + f 3 ( {z}^{′} + β ) . & ( 4 )\end{matrix}$$

Here, the orthogonal slice plane feature value and the parallel slice plane feature value are local maximums at the center position of a vertebra in the direction of z′ axis, and local minimums at the position of an intervertebral disk in the direction of z′ axis. The classifier output feature value is a local maximum at the position of an intervertebral foramen, and a local minimum at a position between intervertebral foramens. Meanwhile, since an anatomical positional relationship between an intervertebral foramen and an intervertebral disk is known, an anatomical positional relationship between the intervertebral foramen and the center position of a vertebra is also known. Therefore, phase difference β for making local maximums agree with each other is added to classifier output feature value f3(z′) in expression (4) to make a position on z′ axis at which the orthogonal slice plane feature value f1(z′) and parallel slice plane feature value f2(z′) are local maximums and a position on z′ axis at which classifier output feature value f3(z′) is a local maximum agree with each other.

Further, feature value f(z′) representing a likelihood of an intervertebral foramen may be calculated by using a fitting function in a similar manner to the second embodiment also in the fourth embodiment. In this case, when a first term and a second term on the right side of expression (4) are added, and represented as feature value f4(z′) collecting orthogonal slice plane feature value f1(z) and parallel slice plane feature value f2(z′), feature value f(z′) representing a likelihood of an intervertebral foramen may be calculated by the following expression (5). In expression (5), a3, a4, b3, b4, c3 and c4 are coefficients for determining the shape of a fitting function, and δ is a phase difference for making a local maximum of orthogonal slice plane feature value f1(z′) and parallel slice plane feature value f2(z′), in other words, a local maximum of feature value f4(z′) agree with a local maximum of classifier output feature value f3(z′).

$$\begin{matrix}[ Expression 5 ] & \\ H ( {a}_{3} , {a}_{4} , {b}_{3} , {b}_{4} , {c}_{3} , {c}_{4} ) = {\sum }_{{z}^{′} = 1}^{{z}^{′}} f 3 ( {z}^{′} ) cos \frac{2 π ( {z}^{′} - {c}_{3} )}{{a}_{3} {z}^{′} + {b}_{3}} + {\sum }_{{z}^{′} = 1}^{{z}^{′}} f 4 ( {z}^{′} ) cos \frac{2 π ( {z}^{′} - {c}_{4} )}{{a}_{4} {z}^{′} + {b}_{4}} + γ . & ( 5 )\end{matrix}$$

In each of the aforementioned embodiments, a search range for calculating a feature value representing a likelihood of an intervertebral foramen is set in a left-side region or a right-side region of the center line 31 set in slice image Di, as illustrated in FIG. 5. Alternatively, search ranges may be set for both of the right-side region and the left-side region, and a feature value representing a likelihood of an intervertebral foramen may be calculated in each of the left-side and right-side search ranges. Here, when feature values calculated in the left-side search range and the right-side search range are fL(z′) and fR(z′), a representative value, such as a maximum value and an average value, of left-side feature value fL(z′) and right-side feature value fR(z′) should be used as a feature value representing a likelihood of an intervertebral foramen in the slice image Di. Feature values fL(z′) and fR(z′) are the first feature value and the second feature value in the present disclosure.

Here, when the feature value representing a likelihood of an intervertebral foramen is fitted to a fitting function as in the second embodiment, a feature value representing a likelihood of an intervertebral foramen that is a representative value of left-side feature value fL(z′) and right-side feature value fL(z′) should be used. Further, as represented by the following expression (6), both of left-side feature value fL(z′) and right-side feature value fR(z′) may be fitted to a fitting function at the same time. In expression (6), al, ar, bl, br, cl and cr are coefficients for determining the shape of a fitting function, and is a weighting coefficient.

$$\begin{matrix}[ Expression 6 ] & \\ H ( {a}_{𝓁} , {a}_{r} , {b}_{𝓁} , {b}_{r} , {c}_{𝓁} , {c}_{r} ) = {\sum }_{{z}^{′} = 1}^{{z}^{′}} f L ( {z}^{′} ) cos \frac{2 π ( {z}^{′} - {c}_{𝓁} )}{{a}_{𝓁} {z}^{′} + {b}_{𝓁}} + {\sum }_{{z}^{′} = 1}^{{z}^{′}} f R ( {z}^{′} ) cos \frac{2 π ( {z}^{′} - {c}_{r} )}{{a}_{r} {z}^{′} + {b}_{r}} + λ D \\ D = {( {a}_{𝓁} - {a}_{r} )}^{2} + {( {b}_{𝓁} - {b}_{r} )}^{2} + {( {c}_{𝓁} - {c}_{r} )}^{2} . & ( 6 )\end{matrix}$$

In the aforementioned embodiments, the center line 30 of the spinal cord is detected. Alternatively, the center line of the spinal column may be detected instead of the center line 30 of the spinal cord. In this case, an anatomical positional relationship between the center line of the spinal column and an intervertebral foramen is fixed on a slice plane orthogonal to the center line of the spinal column. Therefore, it is possible to calculate a feature value representing a likelihood of an intervertebral foramen in a similar to the aforementioned embodiments.

In the aforementioned embodiments, the vertebra identification unit 26 attaches a label to each of identified vertebrae. Alternatively, the vertebra identification unit 26 may only segment plural vertebrae without attaching labels.

What is claimed is:

1. A vertebra segmentation apparatus comprising:
an intervertebral foramen position detection unit that detects positions of intervertebral foramens in a three-dimensional medical image including a plurality of vertebrae; and
a vertebra identification unit that identifies each of the plurality of vertebrae by using the detected positions of the intervertebral foramens, respectively.

2. The vertebra segmentation apparatus, as defined in claim 1, wherein the intervertebral foramen position detection unit detects the positions of the intervertebral foramens by using feature values representing a likelihood of an intervertebral foramen in the three-dimensional medical image.

3. The vertebra segmentation apparatus, as defined in claim 2, the apparatus further comprising:
a center line detection unit that detects at least one of a center line of a spinal cord and a center line of a spinal column in the three-dimensional medical image;
a slice image generation unit that generates, with respect to at least one of the center line of the spinal cord and the center line of the spinal column, at least one slice image for detecting the intervertebral foramens; and
a feature value calculation unit that calculates the feature value representing a likelihood of an intervertebral foramen based on the at least one slice image,
wherein the intervertebral foramen position detection unit detects the positions of the intervertebral foramens based on the feature value.

4. The vertebra segmentation apparatus, as defined in claim 3, wherein the slice image generation unit generates a plurality of slice images about a plurality of slice planes at a predetermined interval that are orthogonal to the center line of the spinal cord or the center line of the spinal column.

5. The vertebra segmentation apparatus, as defined in claim 4, wherein the feature value calculation unit calculates a first feature value representing a likelihood of an intervertebral foramen in one side of each of the plurality of slice images and a second feature value representing a likelihood of an intervertebral foramen in the other side of each of the plurality of slice images, when each of the plurality of slice images is divided in such a manner that the one side and the other side are symmetric with respect to a straight line passing through the center line of the spinal cord or the center line of the spinal column, and determines a representative value of the first feature value and the second feature value, as the feature value representing a likelihood of an intervertebral foramen.

6. The vertebra segmentation apparatus, as defined in claim 3, wherein the slice image generation unit generates the at least one slice image of a slice plane that includes the center line of the spinal cord or the center line of the spinal column and faces a predetermined direction.

7. The vertebra segmentation apparatus, as defined in claim 2, wherein the intervertebral foramen position detection unit fits the feature value to a predetermined periodic function or quasiperiodic function in the direction of the center line of the spinal cord or the center line of the spinal column, and detects the position of each of the intervertebral foramens based on the periodic function or the quasiperiodic function to which the feature value has been fitted.

8. The vertebra segmentation apparatus, as defined in claim 4, wherein the feature value calculation unit calculates a first feature value representing a likelihood of an intervertebral foramen in one side of each of the plurality of slice images and a second feature value representing a likelihood of an intervertebral foramen in the other side of each of the plurality of slice images, when each of the plurality of slice images is divided in such a manner that the one side and the other side are symmetric with respect to a straight line passing through the center line of the spinal cord or the center line of the spinal column, and
wherein the intervertebral foramen position detection unit fits each of the first feature value and the second feature value to a predetermined periodic function or quasiperiodic function in the direction of the center line of the spinal cord or the center line of the spinal column, and detects the position of each of the intervertebral foramens based on the periodic function or the quasiperiodic function to which each of the first feature value and the second feature value has been fitted.

9. The vertebra segmentation apparatus, as defined in claim 2, wherein the intervertebral foramen position detection unit detects the position of each of the intervertebral foramens also by using a feature value representing clearness of a slice plane intersecting the center line of the spinal cord or the center line of the spinal column, a feature value representing clearness of a slice plane parallel to the center line of the spinal cord or the center line of the spinal column and feature values representing regularity of the arrangement of the vertebrae that are calculated based on sharpness of the intersecting slice plane and sharpness of the parallel slice plane.

10. A vertebra segmentation method comprising the steps of
detecting positions of intervertebral foramens in a three-dimensional medical image including a plurality of vertebrae; and
identifying each of the plurality of vertebrae by using the detected positions of the intervertebral foramens, respectively.

11. A non-transitory recording medium having stored therein a vertebra segmentation program that causes a computer to execute the procedures of
detecting positions of intervertebral foramens in a three-dimensional medical image including a plurality of vertebrae; and
identifying each of the plurality of vertebrae by using the detected positions of the intervertebral foramens, respectively.
